(19) United States
(12) Patent Application Publication
(10) Pub. No.: US 2011/0033536 A1
(43) Pub. Date: Feb. 10, 2011

(54) 24-HOUR SUSTAINED-RELEASE METOCLOPRAMIDE

(75) Inventors: John Claude Savoir vilboeuf, Mexico City (MX); Maria Teresa de Jesus Francisco Doce, Mexico City (MX); Teresita del Nino Jesus Costales Gonzalez, Mexico City (MX); Miriam V Illa Vargas, Mexico de Nezahualcoyotl Estado (MX)

Correspondence Address: BROOKS KUSHMAN P.C., 1000 TOWN CENTER, TWENTY-SECOND FLOOR, SOUTHFIELD, MI, 48075, US

(73) Assignee: POSIVISIONARY SOLUTIONS LLP, Bromley, Kent (GB)

(21) Appl. No.: 12/935,187

(22) PCT Filed: Mar. 27, 2009
(86) PCT No.: PCT/MX2009/000026

§ 371 Date: Oct. 20, 2010

(30) Foreign Application Priority Data
Mar. 28, 2008 (MX) MX/A/2008/004268

(51) Int. Cl. A61K 9/36; A61K 31/166; A61P 1/08; A61P 1/04

(52) U.S. Cl. 424/465; 514/619

(57) ABSTRACT

The present invention consists of an extended-release metoclopramide hydrochloride pharmaceutical composition, in 30 mg drug substance 5 tablets, for use in gastrointestinal disorders. The formulation is mainly composed of a hydrophilic polymer, a hydrophobic polymer, a hydrophilic component and metoclopramide hydrochloride. The hydrophilic polymer is swollen by hydration when contacting water, forming a gel coat which controls drug substance release. The water inside the matrix dissolves the drug substance and this is diffused outside through the gel coat. The hydrophobic polymer shows plastic deformation properties under compression, tending to surround the drug substance particles reducing the pore quantity and dimensions in the matrix structure, delaying as a consequence the drug substance release. The hydrophilic component is part of the gel coating structure providing support thereto. Drug substance is the metoclopramide hydrochloride or a pharmaceutically acceptable salt thereof.

### FIELD OF THE INVENTION

The present invention consists of an extended-5 release metoclopramide hydrochloride pharmaceutical composition, in 200 milligram tablets, containing about 30 milligrams of drug substance, for use in gastrointestinal disorders.

### BACKGROUND OF THE INVENTION

Metoclopramide is a wide use compound in gastroenterological practice, its pharmacological effects are evident in gastrointestinal tract (altered gastrointestinal motility and antiemetic effect), even though a prolactin increased secretion and extrapyramidal symptom appearance have been reported. The nature of its gastrointestinal effects has allowed it to be selected as one of the preferred compounds to fight several gastroenterological disorders. Metoclopramide has a pronounced effect on gastrointestinal motility, both in animals and man, administered both orally and intravenously. An increase in esophageal contraction amplitude and a decrease in esophageal sphincter pressure, as well as an increase in amplitude and frequency of antral contractions are included among the metoclopramide effects. Metoclopramide has no effect in gastric secretion. Duodenal contraction is enhanced with the antral contractions within small intestine causing a increase in amplitude of duodenal contractions. Said effects result in a gastric emptiness with a concomitant reduction in transit time within small intestine {Harrington, R. A., et al., 1983, Drugs. Vol. 25: 451-494). Metoclopramide acts by promoting or increasing the intestinal wall coordination enhancing its propulsive activity (Tonini, M. 1996, Pharmacol. Res. Vol. 33: 217-226).

Metoclopramide is indicated for use against esophageal reflux symptoms (Galmiche, J. P., et al. 15 1996. Br. Med. J. Vol 316: 1720-1725. De Caestecker, J. Eur. J. Gastroenter. & Hepatol. 2002. Vol. 14 No. 1:5-7. McCallum, R. W., et al. 1977. New Eng J. Med. Vol. 2 9 6:354-357), which are quite common worldwide. In Spain, annual prevalence of acid reflux is 32%, while in United States a 60% has been reported. (Rey, E., et al. 2006. Rev. Esp. Enf. Dig. Vol. 98, no. 7: 518-526), in Western world, a 20-40% from population is estimated to be affected, with a 7% thereof showing daily symptoms. When this problem is not addressed it may present severe complications including erosive esophagitis, Barret's esophagus and even adenocarcinoma (Pettit, M. 2005. Pharm. World Sci. Vol. 27: 432-435).

Other uses for metoclopramide in medical practice 5 are: peptic ulcer, dyspepsia, anti-emetic (Anthony, L. B., et al. 1986. J. Clin. Oncol. Vol. 4: 98-103) and in digestive system (Thoeni, R. F., & R. G. Wilson, 1988, Radiology, Vol. 16 9:391-393). Metoclopramide is frequently used in gastroparesis (an abnormal gastric motility condition characterized by a slow gastric emptying in absence of any mechanical obstruction. Gastroparesis symptoms include nausea and vomit, an early sensation satiety and abdominal disturbances.

Treatment options for gastroparesis include diet, behavioral changes, prokinetic drugs and surgical interventions (Akheel, S., A. Rattansingh & S Furtado. J. Postgrad. Med. 2005. Vol. 51, No. 1:54-60). Although metoclopramide is a first choice compound in treating several gastric diseases when they reach plasma concentrations above 100 nanograms per milliliter, some undesirable side effects are present, for instance, akathisia (Bateman, D. N., et al. 1979. Br. J. Pharmacol. Vol. 8: 179-182. Parlak, I., et al., 2005. Emer. Med. J. Vol. 22:621-624). Its extended use has been related with psychosis, preferably when drug presentation is in an immediate 5 release form (Lu, M. L., et al. 2002. Ann Pharmacotherapy. Vol. 36, No. 9: 1387-1390).

Extended use has also been related with adverse neurological effects {Grimes, J. D., et al. 1982. Canad Med. Assoc. J. Vol. 126. No. 1:23-25). Its use has also been reported to be associated with severe movement disorders (Ganzini, L., et al. 1993. Arch. Int. Med. Vol. 153: 1469-1475), mainly in long-term use cases (Millar, L. G., and J. Jankovic. 1989. Arch. Int. Med. Vol. 149 Mo. 11:2486-2492).

There are several systems which favor an extended release of a drug substance contained in a drug. Sometimes drug substance is arranged in a tablet core coating this with several coats, thus when the drug passes through the intestinal tract, the most outer tablet coats contact the fluids and drug substance is released. In other cases, such as that in present invention, drug substance is arranged in an inert matrix which slowly releases the drug substance. Following are references of a number of patents which are related with the invention:

U.S. Pat. No. 4,656,024 consists of a slow release 20 mg metoclopramide pharmaceutical 5 composition, having a first metoclopramide coating from 1 to 20% by weight of metoclopramide, from 0.01 to 0.5% by weight of stearic acid and 5 to 15% by weight of talc, and from 2% to 10% by weight of silica gel and sequential coatings of shellac and 10 methacrylate polymer as semi-permeable membrane, being shellac coating from 1 to 10% by weight in total composition.

U.S. Pat. No. 4,780,322, consisting of a slow release metoclopramide pharmaceutical composition, containing sulfonated resins and carboxylic resins.

U.S. Pat. No. 4,808,416 of slow release, sequentially consists of a metoclopramide pharmaceutical composition wherein said drug substance is located in a core; a first ethylacrylate and methylmethacrylate copolymer coating and a second hydroxypropylmethylphtalate cellulose enteric coating.

U.S. Pat. No. 6,770,262 is referred to a method for treatment of gastroparesis, using nasally metoclopramide.

U.S. patent application 2005/0282873 refers to a controlled-release pharmaceutical composition with metoclopramide as drug substance and a hydrophilic polymer, specifically xanthan gum. Traditionally metoclopramide is found in an immediate-release dosage form, which requires a delivery every 8 hours. This dosage form in addition of being complex for patient, involves the risks of reaching plasma concentrations which cause extrapyramidal effects.

One of the objects of present invention is to provide a compound of metoclopramide hydrochloride, or a pharmaceutically acceptable extended release salt thereof, with a lower delivery frequency.

Another object of present invention is to provide a compound of metoclopramide hydrochloride, or a pharmaceutically acceptable extended release salt thereof, which may be administered every 24 hours.

A further objective of present invention is to provide a compound of metoclopramide hydrochloride, or a pharmaceutically acceptable salt thereof, in such a way that being effective but without reaching plasma concentrations which cause extrapyramidal effects.

### BRIEF DESCRIPTION OF THE DRAWINGS

FIG. 1 shows an unit-dose and multiple-dose comparative average plasma profile for 30 mg extended release metoclopramide hydrochloride tablets (LP) and 10 mg immediate release tablets (LI) (Treatment A (N=13), Treatment B (N=13)).

### DETAILED DESCRIPTION OF INVENTION

Present invention provides a medicament for treatment and/or prevention of gastrointestinal disorders, by delivering an effective and/or prophylactic amount of an extended release formulation containing metoclopramide hydrochloride or a pharmaceutically acceptable salt thereof, to any person in need thereof.

Present invention further provides, the use of extended release metoclopramide hydrochloride or a pharmaceutically acceptable salt thereof, for treatment and/or prevention of gastrointestinal disorders.

A procedure for formulation manufacturing is below provided, including but not limited to:

1. Drug substances and excipients are provided.

2. Drug substance and excipients are screened in order to remove lumps.

3. Components are mixed and the mixture is 5 compressed to a 100 mg preferred weight.

4. Tablets are conditioned in packaging material.

5. Manufacturing process and used equipments are those of conventional use for manufacturing a drug product with above features. The formulation is mainly composed of:

a) A hydrophilic polymer which is swollen by hydration upon contacting water forming a gel coating controlling a release of drug substance. The water within the matrix dissolves the drug substance and this is externally diffused through the gel coating. The hydrophilic polymer is selected from a number of products, including: methylcellulose, hydroxyethylcellulose, hydroxypropylcellulose and hydroxypropylmethylcellulose. On the other hand, the polymer is overhydrated on the matrix surface upon solubilization, being present a matrix wear out as a consequence of an erosion mechanism.

b) A hydrophobic polymer, showing plastic deformation under compression and tending to surround the drug substance particles, reducing the pore quantity and dimensions in the matrix structure and consequently delaying a release of drug substance.

c) Hydrophobic polymer is selected from a plurality of products such as: ethylcellulose, glycerylmonostearate and fatty acids such as acetyl tributyl citrate.

d) An hydrophilic component, showing a synergic effect with the hydrophilic polymer and forming part of the gel coating structure providing support thereto, contributing as a consequence to the control of the drug substance release. Hydrophilic component is selected from a plurality of products such as, cross-linked binding sodium carboxymethylcellulose, cross-linked binding polyvinylpyrrolidone, sodium glycolate starch, pregelatinized starch and modified cellulose.

e) The drug substance, metoclopramide hydrochloride or a pharmaceutically acceptable salt thereof.

The formulation is designed to be delivered every 24 hours.

Description of Formulation Component Performance

Hydrophilic matrices result from compressing a hydrophilic polymer with a drug substance of certain solubility. The hydrophilic polymer is swollen by hydration decreasing the drug substance release ratio up to a fixed or theoretically constant value. Drug substance release depends on the diffusion capability through the polymeric net, the matrix erosion capability or a combination of both processes.

In the hydrophilic matrix developed for present invention, release is controlled when the water soluble polymer is rapidly hydrated on the tablet surface to form a gel coat, which controls water penetration into said tablet. Water inside dissolves the drug substance and this is diffused through the net formed by the gel. Gel coat strength is controlled by polymer viscosity and concentration.

The water-insoluble hydrophobic polymer controls the drug substance release modifying the diffusion path size and length. Although the polymer is water insoluble, it may collect water due to the shown capability in forming hydrogen bridges with water.

The polymer shows plastic deformation properties under compression, tending to surround the drug substance particles, reducing the number of pores in the matrix structure contributing to drug substance release control.

The component related with water, which is swollen when being in contact with, contributes to gel formation through a synergic interaction with the water-soluble polymer, being part of the gel structure. This condition allows obtaining tablets with reproducible dissolution profiles.

Comparative Bioavailability of 30 mg Metoclopramide Hydrochloride Tablets and 10 mg Immediate Release Tablets in Healthy Male subjects.

A multiple-dosage, open, parallel, randomized, clinical trial of comparative pharmacokinetics was carried out for two oral delivery metoclopramide hydrochloride formulations, in 26 healthy male 15 subjects between 18 and 55 years in order to determine pharmacokinetics profiles, establishing and comparing bioavailability as well as assessing safety and tolerance for two 30-mg extended-release metoclopramide hydrochloride tablet formulations and one 10-mg immediate-release metoclopramide hydrochloride tablet formulation.

The 10-mg immediate-release metoclopramide hydrochloride tablets were administered every 8 hours and those of 30-mg extended-release were administered every 24 hours.

Volunteers were randomly assigned to each treatment, which was orally administered swallowing with 250 ml water. For the single-dose pharmacokinetics study, blood samples were collected on times: 0 h (pre-dosage), 0.5 h, 1.0 h, 1.5 h, 2.0 h, 3.0 h, 4.0 h, 6.0 h, 8.0 h, 10.0 h, 12.0 h and 24.0 h for treatment A, and on times 0 h (pre-dosage), 0.5 h, 1.0 h, 1.5 h, 2.0 h, 3.0 h, 4.0 h, 6.0 h, 8.0 h, for treatment B. For multiple-dose pharmacokinetics study, blood samples were collected for both treatment groups on times 0 h (pre-dosage), 0.5 h, 1.0 h, 1.5 h, 2.0 h, 3.0 h, 4.0 h, 6.0 h, 8.0 h, 10.0 h, 12.0 h and 24.0 h after last dose.

Collected plasma for each blood sample was kept under freezing conditions (˜40° C.) until analysis. Unaltered metoclopramide in plasma samples was quantified with a validated method for High Resolution Liquid Chromatography (HPLC) with fluorescence detection.

Results

Single Dose and Multiple Dose Pharmacokinetics

Based on quantified concentrations of unaltered metoclopramide in plasma samples from single-dose and multiple-dose administration from both treatments, pharmacokinetics parameters were calculated which describe metoclopramide pharmacokinetics and bioavailability. Pharmacokinetics parameter calculations were performed by a non-compartment method and they are reported in Tables 1 and 2.

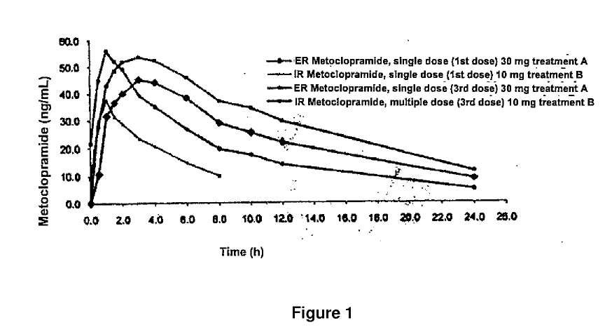

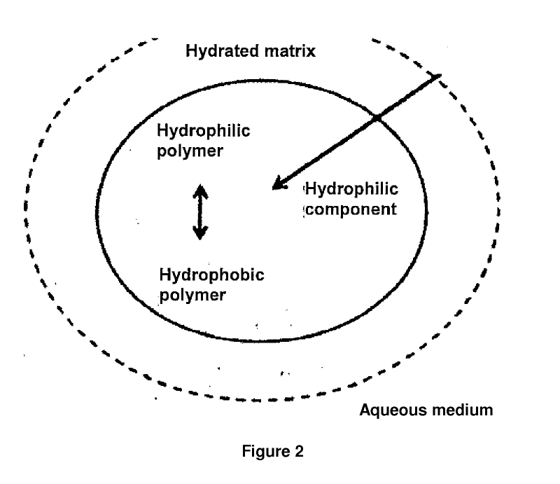

Plasma profile graphs for metoclopramide concentration versus time, for single dose (day 0) and multiple dose (days 2 and 3) administrations, in an arithmetic and semi-logarithmic scale are represented in FIG. 1.

TABLE 1
Pharmacokinetic parameters after a first
administration of 30-mg extended-release
metoclopramide tablets and 10-mg immediate-release tablets.

| | Treatment A (30 mg | | | Treatment B | | |
|---|---|---|---|---|---|---|
| | Tablets) n = 13 | | | (10 mg) n = 13 | | |
| | Arithmetic | | | Arithmetic | | |
| Pharmacokinetic | average ± | | [symbol] | average ± | | [symbol] |
| parameter | S.D. | [symbol] .V. [symbol] | [symbol] | S.D. | [symbol] . [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |

Treatment A: Extended-release metoclopramide hydrochloride tablets, 30 mg every 24 hours.
Treatment B: Immediate-release metoclopramide hydrochloride tablets, 10 mg every 8 hours. [symbol]
indicates data missing or illegible when filed

TABLE 2
Pharmacokinetic parameters after the last
administration of 30-mg extended-release metoclopramide
tablests and 10-mg immediate-release tablets.

| | Treatment A (30 mg | | | Treatment B (10 mg- | | |
|---|---|---|---|---|---|---|
| | Tablets-Third dose = 90 mg) | | | Seventh dose = 70 mg) | | |
| | n = 13 volunteers | | | n = 13 volunteers | | |
| | Arithmetic | | | Arithmetic | | |
| Pharniacokinetic | average ± | C.V. | Minimum | average ± | C.V. | Minimum |
| parameter | S.D. | (%) | Maximum | S.D. | (%) | Maximum |
| ABC72-96 | 747.50 ± 231.43 | 31.0 | 448.20-1202.91 | 463.91 ± 192.55 | 41.5 | 140.67 |
| (hr * ng/mL} | | | | | | 881.98 |
| ABC72-INF | 913.35 ± 319.85 | 35.0 | 513.37-1586.75 | 530.50 ± 238.48 | 45.0 | 151.00 |
| (hr * ng/ml) | | | | | | 1115.96 |
| % ABCextra | 16.68 ± 7.88 | 47.3 | 6.28-33.10 | 11.82 ± 5.74 | 48.5 | 2.89-22.75 |
| (%) | | | | | | |
| Cmax | 57.0982 ± 15.5062 | 27.2 | 32.4886 | 60.1607 ± 17.2432 | 28.7 | 33.8665 |
| (ng/mL} | | | 86.7560 | | | 84.8332 |
| Tmax (hr) | 2.92 ± 1.19 | 40.6 | 1.00-6.00 | 1.04 ± 0.43 | 41.5 | 0.50-2.00 |
| Ke (1/hr) | 0.0822 ± 0.0224 | 27.2 | 0.0479-0.1200 | 0.1038 ± 0.0409 | 39.4 | 0.0625 |
| | | | | | | [symbol] |

Treatment A: Extended-release metoclopramide hydrochloride tablets, 30 mg every 24 hours.
Treatment B: Immediate-release metoclopramide hydrochloride tablets, 10 mg every 8 hours. [symbol]
indicates data missing or illegible when filed

Characterization of Extended Release Metoclopramide Tablets Type

Based on pharmacokinetic parameter results shown in Tables 1 and 2, and on the considerations for characterization of an extended- or controlled-release product (Blume, Gundert & R. Molly 1991. Modified release product. Wisenchaftliche Verlagegesellschaf GmbH. Stutgart), extended-release metoclopramide hydrochloride tablets show slow release kinetics, since Cmax is lower than that for immediate-release drug product; further a Tₘₐₓ of about 3.0 h in the extended-release product was observed, regarding to Tmax of 1 h for immediate-release drug product. Clearance half-life was not modified between both drug products.

There is not a clearly defined concentration therapeutic range for clinical response and toxicity; however a clear relationship has been determined between extrapyramidal effects {akathisia) and plasma concentrations above 120 ng/ml and as noticed, both in Tables 1 and 2 and in plasma concentration graphs, none of the observed Cmax in single-dose and multiple-dose administration is above or close in variability to this 120 ng/ml concentration.

Average plasma concentration (Cavg) obtained from both the first dosage and the last dosage from 30 mg extended-release product was 23.9 and 31.15 ng/ml respectively and when compared with those obtained 5 with 10 mg immediate-release product (20.64 and 35.59 ng/ml) did not show statistically significant differences (p>0.05). In the light of the above, extended-release tablets show the same average concentrations than the immediate-release product, 10 but with the advantage of only one dose in 24 hours and lower concentration fluctuations along 24 hours as observed with immediate-release product.

### CONCLUSIONS

### Pharmacokinetics and Bioavailability

Metoclopramide Plasma Profiles for Single-Dose

{1^{st} dose) and multiple-dose (3^{rd} dose) extended-release formulations, showed a delayed tmax more than 3 hours (1.00-6.00 h) compared with immediate release {0.50-2.00 h).

Clearance half-life (tH) for 30 mg extended release tablets {9.23 h) was approximately double than observed in 10 mg immediate release tablets (4.06 mg). In multidose administration (day 3), tM. for 30 mg extended release tablets and 10 mg immediate release tablets (9.03 h and 7.43) did not show significant differences (p>0.05).

Average plasma concentration (Cavg) in the first administration showed plasma levels higher than 5 ng/ml from 0.5 to 24 hours for treatment A (extended release) and 0.5 to 8 h for treatment B (Plasil®) in single dose administration.

Average plasma concentration (Cavg), both in the first dose and in the last dose in both study 10 treatments with the corresponding dose and posology did not show statistically significant differences (p>0.05). The average concentrations of mg extended release product being similar with 10 mg immediate release product, the efficiency profile may 15 be considered equivalent.

Metoclopramide product (treatment A), 30 mg extended-release tablets, may be considered as a “extended release and slow release” product since Cmax is decreased, tmax is delayed and clearance 20 half-life is not modified relative to the immediate release product.

Following are examples of different optimum formulations to obtain metoclopramide hydrochloride extended release tablets to a 30 mg dose per tablet.

### Example 1

TABLE 2
Pharmacokinetic [symbol]
administration of 30-mg exten [symbol]
tablets and 10-mg immediate-releas

[symbol]

| | Treatment A (30 mg | | | Treatment B (10 mg- | | |
|---|---|---|---|---|---|---|
| | Tablets-Third dose = 90 mg) | | | Seventh dose = 70 mg) | | |
| | n = 13 volunteers | | | n = 13 volunteers | | |
| | Arithmetic | | | Arithmetic | | |
| Pharniacokinetic | average ± | C.V. | Minimum | average ± | C.V. | Minimum |
| parameter | S.D. | (%) | Maximum | S.D. | (%) | Maximum |
| ABC72-96 | 747.50 ± 231.43 | 31.0 | 448.20-1202.91 | 463.91 ± 192.55 | 41.5 | 140.67 |
| (hr * ng/mL} | | | | | | 881.98 |
| ABC72-INF | 913.35 ± 319.85 | 35.0 | 513.37-1586.75 | 530.50 ± 238.48 | 45.0 | 151.00 |
| (hr * ng/ml) | | | | | | 1115.96 |
| % ABCextra | 16.68 ± 7.88 | 47.3 | 6.28-33.10 | 11.82 ± 5.74 | 48.5 | 2.89-22.75 |
| (%) | | | | | | |
| Cmax | 57.0982 ± 15.5062 | 27.2 | 32.4886 | 60.1607 ± 17.2432 | 28.7 | 33.8665 |
| (ng/mL} | | | 86.7560 | | | 84.8332 |
| Tmax (hr) | 2.92 ± 1.19 | 40.6 | 1.00-6.00 | 1.04 ± 0.43 | 41.5 | 0.50-2.00 |
| Ke (1/hr) | 0.0822 ± 0.0224 | 27.2 | 0.0479-0.1200 | 0.1038 ± 0.0409 | 39.4 | 0.0625 |
| | | | | | | 0.2111 |
| T Hel (hr) | 9.03 ± 2.49 | 27.5 | 5.78-14.47 | 7.43 ± 2.21 | 29.8 | 3.28-11.09 |
| Cavg | 31.1459 ± 9.6431 | 31.0 | 18.6752 50.1211 | 35.5881 ± 12.2903 | 34.5 | 15.0010 |
| (ng/mL) | | | | | | 60.0748 |

| | QUANTITY |
|---|---|
| COMPONENT | mg/tablet |
| Metoclopramide Hydrochloride | 30.00 |
| Corn starch | 83.50 |

[symbol]

indicates data missing or illegible when filed

1. An extended release pharmaceutical composition, a tablet of about 200 milligrams, for release into the gastrointestinal environment, comprising metoclopramide hydrochloride from about 21 to 35 milligrams by weight, from hydrophilic and hydrophobic polymers and hydrophilic components and hydrophilic components which promote water penetration within the tablet, all those from about 179 to 165 milligrams by weight, which are pharmaceutically acceptable so that when composition is orally taken, extended release is induced while keeping a bioavailability substantially equivalent to the immediate release composition.

2. Extended release pharmaceutical composition according to claim 1, characterized in that comprises hydrophilic, hydrophobic polymers as well as hydrophilic component which promote water penetration within the tablet.

3. Extended release pharmaceutical composition according to claim 1, characterized in that the hydrophilic polymer is selected from the group consisting of methylcellulose, hydroxyethylcellulose, hydroxypropylcellulose and hydroxypropylmethylcellulose.

4. Extended release pharmaceutical composition according to claim 1, characterized in that the hydrophilic component promoting water penetration inside the tablet is selected from the group consisting of crosslinked binding sodium carboxymethylcellulose, crosslinked binding polyvinylpyrrolidone, sodium glycolate starch, pregelatinized starch and modified cellulose.

5. Extended release pharmaceutical composition according to claim 1, characterized in that the extended release pharmaceutical composition in gastrointestinal environment, comprises a hydrophilic polymer, a hydrophobic polymer and a hydrophilic component in a percentage of about 179 to 165 milligrams by weight.

6. Extended release pharmaceutical composition according to claim 1, characterized in that the hydrophilic polymer is methylcellulose.

7. Extended release pharmaceutical composition according to claim 1, characterized in that the hydrophilic polymer is hydroxyethylcellulose.

8. Extended release pharmaceutical composition according to claim 1, characterized in that the hydrophilic polymer is hydroxypropylcellulose.

9. Extended release pharmaceutical composition according to claim 1, characterized in that the hydrophilic polymer is hydroxypropylmethylcellulose.

10. Extended release pharmaceutical composition according to claim 1, characterized in that the hydrophobic polymer is selected from the group consisting of ethylcellulose, glyceryl monostearate and fatty acids such as acetyl tributyl citrate.

11. Extended release pharmaceutical composition according to claim 1, characterized in that the hydrophobic polymer is ethylcellulose.

12. Extended release pharmaceutical composition according to claim 1, characterized in that the hydrophobic polymer is glyceryl monostearate.

13. Extended release pharmaceutical composition according to claim 1, characterized in that the hydrophobic polymer is a fatty acid such as acetyl tributyl citrate.

14. Extended release pharmaceutical composition according to claim 1, characterized in that the hydrophilic component is selected from the group consisting of crosslinked binding sodium carboxymethylcellulose, crosslinked binding polyvinylpyrrolidone, sodium glycolate starch, pregelatinized starch and modified cellulose.

15. Extended release pharmaceutical composition according to claim 1, characterized in that the hydrophilic component promoting water penetration inside the tablet is sodium carboxymethylcellulose.

16. Extended release pharmaceutical composition according to claim 1, characterized in that the hydrophilic component promoting water penetration inside the tablet is polyvinylpyrrolidone.

17. Extended release pharmaceutical composition according to claim 1, characterized in that the hydrophilic component promoting water penetration inside the tablet is sodium glycolate starch.

18. Extended release pharmaceutical composition according to claim 1, characterized in that the hydrophilic component promoting water penetration inside the tablet is pregelatinized starch.

19. Extended release pharmaceutical composition according to claim 1, characterized in that the hydrophilic component promoting water penetration inside the tablet is modified cellulose.

20. Extended release pharmaceutical composition according to claim 1, characterized in that creates an increase in peristaltic movement amplitude in esophagus, gastric antrum and small intestine and an increase in propulsive motility from gastrointestinal content.

21. Extended release pharmaceutical composition according to claim 1, characterized in that comprises about 30 mg of metoclopramide hydrochloride or a pharmaceutically acceptable salt thereof.

22. Extended release pharmaceutical composition according to claim 1, characterized in that is administered for treatment or prevention of disorders such as: vomit, esophageal gastric reflux and nausea.

23. Extended release pharmaceutical composition according to claim 1, characterized in that the metoclopramide hydrochloride formulation or a pharmaceutically acceptable salt thereof reduce the likelihood of reaching plasma concentrations which generate extrapyramidal effects.

24. Extended release pharmaceutical composition according to claim 1, characterized in that the metoclopramide hydrochloride formulation or a pharmaceutically acceptable salt thereof show a lower frequency in administration.

25. Extended release pharmaceutical composition according to claim 1, characterized in that the metoclopramide hydrochloride formulation or pharmaceutically acceptable salt thereof is administered every 24 hours.
